(19) United States
(12) Patent Application Publication
(10) Pub. No.: US 2025/0344997 A1
(43) Pub. Date: Nov. 13, 2025

(54) SENSOR DATA COMMUNICATION USING ECG INTERFACE

(71) Applicant: University of Washington, Seattle, WA (US)

(72) Inventors: Anandghan Waghmare, Seattle, WA (US); Shwetak N. Patel, Seattle, WA (US)

(21) Appl. No.: 19/203,041

(22) Filed: May 8, 2025

Related U.S. Application Data
(60) Provisional application No. 63/645,654, filed on May 10, 2024.

(51) Int. Cl. A61B 5/00; A61B 5/0205; A61B 5/08; A61B 5/256; A61B 5/28; A61B 5/332; A61B 5/333

(52) U.S. Cl. CPC A61B 5/681; A61B 5/0006; A61B 5/02055; A61B 5/256; A61B 5/28; A61B 5/332; A61B 5/082; A61B 5/333; A61B 2560/0242; A61B 2560/0468; A61B 2562/164; A61B 2562/166

(57) ABSTRACT

An example method involves providing data from an external sensor device to an electrocardiogram (ECG) application, and then decoding the ECG application's output to obtain the original sensor data. Systems described herein include external sensor devices which may provide sensor data to electronic devices, such as smartwatches, through an ECG interface.

### CROSS-REFERENCE TO RELATED APPLICATIONS

This application claims the benefit under 35 U.S.C. § 119 of the earlier filing date of U.S. Provisional Application Ser. No. 63/645,654 filed May 10, 2024, the entire contents of which are hereby incorporated by reference in their entirety for any purpose.

### TECHNICAL FIELD

Examples described herein relate generally to electronic devices including external sensors. Examples of the use of ECG hardware as a communication interface for an external sensor are described.

### BACKGROUND

Wearable devices, such as ubiquitous smartwatches, are becoming integral to our modern lifestyle and assessments of health. Central to their functionality is often an array of sensors, each designed to capture a specific aspect of the wearer's environment or physiological state. From tracking heart rate and sleep patterns to monitoring physical activity and environmental exposure, these wearables gather a wealth of information that offer insights into an individual's well-being.

It can be limiting to be restricted to the fixed sensor set of commercial electronic devices, such as smartwatches, when a different or specialized sensor would be desirable for a particular user purpose.

Single-function devices that contain specialized sensors along with a dedicated processor, communication stack, and wearable housing hardware, would be expensive. The likelihood of incorporating these specialized sensors into mass produced smartwatches is low due to increased production costs, integration complexity, and potentially limited market appeal for highly specific features.

### BRIEF DESCRIPTION OF THE DRAWINGS

FIG. 1 is a schematic illustration of a system, arranged in accordance with examples described herein.

FIG. 2 is a schematic illustration of electrodes and ECG circuitry, arranged in accordance with examples described herein.

FIG. 3 is a schematic illustration of watches arranged in accordance with examples described herein.

FIG. 4 is a schematic illustration of an external sensor device arranged in accordance with examples described herein.

FIG. 5A is a schematic illustration of a system arranged in accordance with examples described herein.

FIG. 5B is a schematic illustration of a system arranged in accordance with examples described herein.

FIG. 5C is a schematic illustration of a system arranged in accordance with examples described herein.

FIG. 6A is a schematic illustration of a system arranged in accordance with examples described herein.

FIG. 6B is a schematic illustration of a system arranged in accordance with examples described herein.

FIG. 7A is a schematic illustration of a system arranged in accordance with examples described herein.

FIG. 7B is a schematic illustration of a system arranged in accordance with examples described herein.

FIG. 7C is a schematic illustration of a system arranged in accordance with examples described herein.

FIG. 8A is a schematic illustration of a system arranged in accordance with examples described herein.

FIG. 8B is a schematic illustration of a system arranged in accordance with examples described herein.

FIG. 9 is a flowchart of a method arranged in accordance with examples described herein.

### DETAILED DESCRIPTION

Certain details are set forth herein to provide an understanding of described embodiments of technology. However, other examples may be practiced without various of these particular details. In some instances, well-known circuits, control signals, sensors, ECG operations, timing protocols, and/or software operations have not been shown in detail in order to avoid unnecessarily obscuring the described embodiments. Other embodiments may be utilized, and other changes may be made, without departing from the spirit or scope of the subject matter and/or claims presented here.

Wearable manufacturers generally negotiate the marketability versus utility trade-off by catering their designs to broad market demands, not the needs of more niche audiences, which can vary considerably. For instance, individuals who spend significant time outdoors, such as field researchers or construction workers, could greatly benefit from a wearable device equipped with an ultraviolet (UV) light sensor; such a device would help them monitor and log their exposure to harmful UV radiation, thereby aiding in the prevention of skin-related health issues. Similarly, people living in areas with high air pollution might find value in wearables that can monitor air quality across different locations, alerting them to potentially hazardous environmental conditions. Further, certain functions can be very useful as specific times, such as a breathalyzer sensor after drinking, but not necessarily something one would need all the time. Those with specific motor abilities may prefer physical buttons for specific watch functions over the touchscreen.

Examples described herein include examples of communication techniques that may allow electronic devices (e.g., smartwatches) to leverage existing electrocardiogram (ECG) hardware as a data communication interface. Examples may allow the connection of external sensors, expanding electronic device functionality. This may advantageously cater to specialized user needs beyond those offered by pre-built sensor suites. Example systems include external sensors coupled to ECG hardware may be able to be priced at a fraction of the cost and may consume a fraction of the power of traditional communication protocols, such as Bluetooth Low Energy. Cost-effective add-on sensors described herein may leverage the existing display, data logging, and processing power of electronic devices such as smartwatches. The use of low-power designs in some examples may further enhance the user experience by reducing the need for frequent charging.

Examples described herein include an approach that leverages electrocardiogram (ECG) hardware on electronic devices, such as smartwatches, for receiving data from external sensors. The use of the ECG hardware to receive external sensor data may allow for low-power communication with minimal and inexpensive components.

Generally, external sensors may transmit sensor data encoded in voltage variations through frequency modulation, allowing the ECG hardware to capture the sensor data. The ECG interface is generally engineered to identify voltage potentials—e.g., the tiny electrical signals generated by the heart. Compared to traditional methods like Bluetooth Low Energy (BLE) and WiFi, examples of this method of sensor data communication may offer significant advantages useful for add-on sensors: examples may consume more than two orders of magnitude less power during active communication and use a handful inexpensive modulation components costing about ten times less than a BLE solution.

Moreover, use of wireless protocols would involving having an add-on sensor also contain or utilize its own microcontroller, radio, analog sensing front end, analog-to-digital converter (ADC), and associated power management; these increase cost, power, and complexity over a wired solution. By leveraging the ECG front-end, examples described herein may obviate and/or reduce the need for these components.

An implemented example utilizes a low-dropout voltage regulator (LDO) TPS7A 0318PDBVR, $0.118/unit) and a 555 timer (ICM 7555IBAZ-T, $0.010/unit) for a total cost under $0.13. This combination consumes less than 20 μW during active transmission. In comparison, Bluetooth Low Energy (BLE) communication with the NRF52805 chip (from $1.214/unit) consumes about 5.7 mW during active transmission (datasheet from Nordic-semi.com). Component costs described herein are sourced from Octopart.com (on a 10,000-unit basis).

In this manner, niche add-on sensors may significantly enhance the usefulness of an electronic device, such as a smartwatch or other wearable device, for specific needs. Tested implemented examples were used to characterize the ECG interface as a communication channel on commercial smartwatches.

Implemented examples include four different sensors that use both continuous background and on-demand sensing: a UV light sensor, a body temperature sensor, external buttons for smartwatch input, and a breath alcohol sensor. Other sensors may be used in other examples. These sensors were implemented in communication with smartwatches—e.g., Apple Watch Series 9 and Google Pixel Watch 2—via ECG, showcasing examples of the communication methodology described herein. The external sensors may be packaged in a variety of form factors (e.g., bands or cases) and may utilize a variety of power sources (e.g., self-powered or primary batteries).

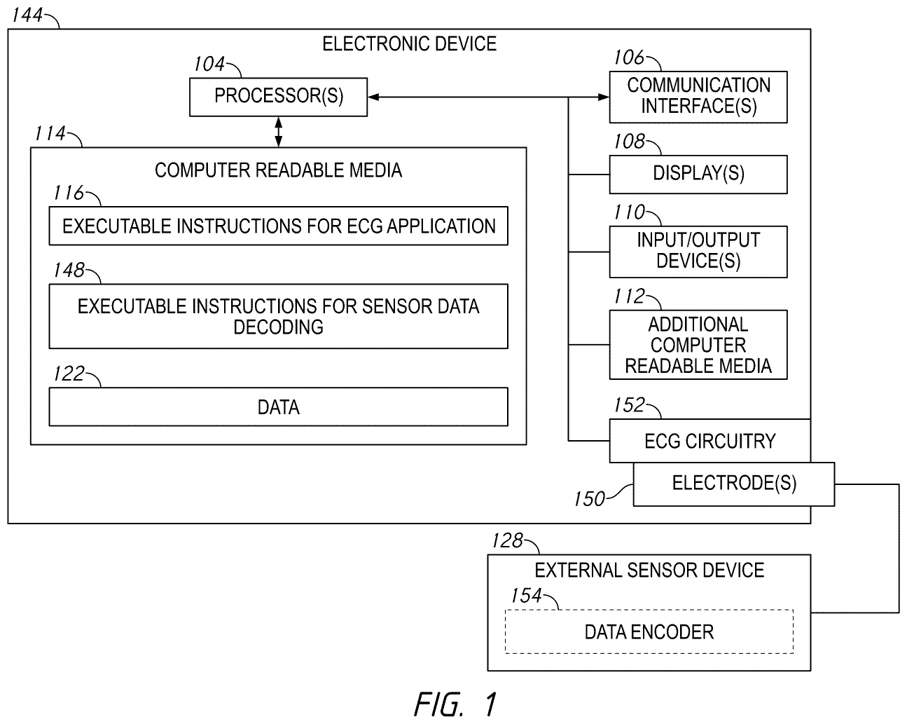

FIG. 1 is a schematic illustration of a system arranged in accordance with examples described herein. The system of FIG. 1 includes electronic device 144 and external sensor device 128. The electronic device 144 may include processor(s) 104, computer readable media 114, communication interface(s) 106, display(s) 108, input/output device(s) 110, additional computer readable media 112, and ECG circuitry 152 including electrode(s) 150. The computer readable media 114 may include executable instructions for ECG application 116, executable instructions for sensor data decoding 148, and data 122.

FIG. 1 is an exemplary system depiction. Additional, fewer, and/or different components may be used in other examples.

Electronic devices described herein, such as the electronic device 144 of FIG. 1 may be implemented using one or more wearable devices. Examples of wearable devices include, but are not limited to, smartwatches, rings, anklets, visors, helmets, socks, necklaces, headbands, eyeglasses, goggles, and/or medical devices. In some examples, the electronic device 144 may be implemented using a smartwatch such as an APPLE watch series 9 or a GOOGLE PIXEL Watch 2. Generally, electronic devices described herein may include components configured to obtain an ECG reading from a user, such as executable instructions for ECG application 116 and ECG circuitry 152.

Electronic devices, such as the electronic device 144 of FIG. 1, may include one or more processors, such as the processor(s) 104. Any kind and/or number of processors may be present, including one or more central processing unit(s) (CPUs), graphics processing units (GPUs), other computer processors, processor cores, mobile processors, digital signal processors (DSPs), field programmable gate arrays (FPGAs), application specific integrated circuits (ASICs), microprocessors, controllers and/or processing units configured to execute machine-language instructions and process data.

Electronic devices, such as the electronic device 144 of FIG. 1, may further include computer readable media, such as computer readable media 114. Any type or kind of media may be present, including memory and/or storage. Examples include read only memory (ROM), random access memory (RAM), solid state drive (SSD), secure digital card (SD card), hard drive, network-attached storage, etc. While a single schematic square is depicted as computer readable media 114 in FIG. 1, any number of memory and/or storage devices may be present, and the executable instructions and/or data shown may be stored on one or more separate devices in some examples. The computer readable media may be in communication (e.g., electrically connected) to the processor(s), such as computer readable media 114 is coupled to processor(s) 104.

The computer readable media may store executable instructions for execution by the processor(s), such as executable instructions for ECG application 116. The executable instructions for ECG application 116 may be executed by one or more processor(s) 104 to output ECG data for a user. The executable instructions for ECG application 116 may, for example, obtain signals from ECG circuitry 152 coupled to electrode(s) 150. In this manner, ECG data for a user may be obtained. The executable instructions for ECG application 116 may accordingly output signals received from ECG circuitry 152.

Note generally that Electrocardiogram EKG) generally refers to a medical technique that captures and records the heart's electrical activity over time. This method may be used in diagnosing and monitoring heart conditions by capturing the electrical patterns of the heart, which indicate how its muscles contract and pump blood. Performing an ECG typically involves placing electrodes on the patient's skin at locations around the chest, arms, and legs. These electrodes sense the minute electrical changes on the skin caused by the heart muscle's activity during each heartbeat. Medical ECG procedures may use as many as ten electrodes, providing 12 different views (leads) of the heart's activity, each offering unique insights into the heart's health and functioning.

ECG circuitry 152 may include electrodes, amplifiers, filters, and/or other components. Amplifiers are generally used for boosting the heart's faint electrical signals to a level that can be processed and visualized. The heart's electrical signals are typically weak and can vary from 1 μV to 100 mV, with a typical value of 1 mV. Filters may be used to help eliminate noise and interference, ensuring the signals accurately represent the heart's activity. The captured signals are then digitally processed (e.g., using processor(s) 104 and/or other circuitry) to enhance the signal-to-noise ratio (SNR), ensuring clearer and more accurate readings. Accordingly, the ECG circuitry 152 may amplify and/or filter a signal provided from one or more electrode(s) 150. A voltage indicative of heart signals may be expected at electrode(s) 150, however, in examples described herein, other sensor data may be provided to electrode(s) 150.

Generally, the ECG circuitry 152 may be configured to measure low-amplitude heart signals. Accordingly, the ECG circuitry 152 may have a high gain to allow the heart signals to be detected. Examples described herein utilize the ECG circuitry 152 for information transmission from external sensors. By encoding external sensor data as voltage (or other signal parameter) fluctuation, the fluctuations can be detected by the ECG circuitry 152 and detected as described herein. In this manner, the ECG circuitry 152 may be suitable not only for heart rate monitoring but also for communication of data as described herein.

In some examples, the ECG circuitry 152 may include one or more analog-to-digital converters or other circuitry which may generate a digital signal corresponding to an analog input from one or more ECG electrodes. The digital data stream may be provided to an ECG application. Accordingly, the executable instructions for ECG application 116 may include instructions for receiving a data stream indicative of an analog input to one or more ECG electrodes.

The computer readable media may store executable instructions for execution by the processor(s), such as executable instructions for sensor data decoding 148. The executable instructions for sensor data decoding 148 may be executed by one or more processor(s) 104 to decode sensor data which may be represented by an output of the ECG application in accordance with examples described herein. For example, the executable instructions for ECG application 116 may output ECG data. However, in some examples described herein an output of the executable instructions for ECG application 116 may additionally or instead represent data received from an external sensor device.

Accordingly, the executable instructions for sensor data decoding 148 may include instructions for decoding the data (e.g., demodulating and/or otherwise post-processing an output of the executable instructions for ECG application 116). In some examples, the executable instructions for sensor data decoding 148 may include instructions for obtaining an output data stream from the ECG circuitry. The output data stream may be obtained, for example, from an ECG application, such as in accordance with executable instructions for ECG application 116. While executable instructions for sensor data decoding 148 are shown and described, hardware (e.g., circuitry) for decoding may additionally or instead be provided. Moreover, while the executable instructions for sensor data decoding 148 are shown as incorporated in electronic device 144, it is to be understood that the sensor data may be communicated from the electronic device 144 to one or more other computer systems (e.g., using wired or wireless communication). The executable instructions for sensor data decoding 148 and/or decoding circuitry may be present at the receiving computer system to decode and/or further analyze the sensor data.

In this manner, electronic devices described herein (such as smartwatches or other wearable devices) may include an ECG application—e.g., software for generating a digital data stream from an analog input to one or more ECG electrodes. Such an application may be represented in FIG. 1 as executable instructions for ECG application 116. Electronic devices described herein may include a sensor data decoding application. The sensor data decoding application may be represented in FIG. 1 as executable instructions for sensor data decoding 148. The sensor data decoding application may obtain a data stream from the ECG application and decode the sensor data from the ECG application output.

The computer readable media 114 and/or other computer readable media accessible to the electronic device 144 may store data, such as data 122. The data 122 may include external sensor data, which may have been decoded in accordance with executable instructions for sensor data decoding 148. The data 122 may include ECG data which may be output from the executable instructions for ECG application 116. Additional, different, and/or other data may be present.

The electronic device of FIG. 1 may include additional components, not all of which are necessarily depicted in FIG. 1. Example of additional components may include one or more communication interface(s), such as communication interface(s) 106. The communication interface(s) 106 may include as a WiFi, Ethernet, Bluetooth, network interface, cellular interface and/or other communications interface(s). The electronic device 144 may include one or more display(s), such as display(s) 108. The display(s) 108 may display ECG data described herein and/or decoded external sensor data.

The electronic device 144 may include one or more input and/or output devices, such as input/output device(s) 110 including, but not limited to, one or more touchscreens, mice, keyboards, and/or cameras. The electronic device may include and/or be in communication with additional computer systems.

Examples of systems described herein may include one or more external sensor devices, such as external sensor device 128 of FIG. 1. The external sensor device 128 may, for example, be implemented using a UV light sensor, a body temperature sensor, a breath alcohol sensor, and/or a touch button. Other sensor devices may be used in other examples. The external sensor device may include its own data generation circuitry and/or other circuitry.

Examples of systems described herein may advantageously utilize communication between an external sensor device, such as external sensor device 128 of FIG. 1 and a computer system, such as electronic device 144 of FIG. 1 through an ECG application. Accordingly, data generated by an external sensor device may be provided to one or more electrodes utilized by ECG circuitry, such as to the electrode(s) 150 coupled to the ECG circuitry 152 of FIG. 1.

The external sensor device may be implemented using a form factor which may facilitate coupling between the external sensor device and an input to an ECG application (e.g., to one or more electrodes). For example, the external sensor device 128 may be implemented using a flexible circuit, case, attachment, band wrap, or other form factors which may allow for the external sensor device 128 to be positioned and/or attached to the electronic device 144. In some examples, the external sensor device 128 may be positioned such that one or more output electrodes of the external sensor device 128 contact the electrode(s) 150. In some examples, the external sensor device 128 may be positioned such that a user may contact (e.g., using one or more fingers) both the external sensor device 128, such as an electrode of the external sensor device 128, and the electronic device 144, such as the electrode(s) 150 and/or another ground node for the electronic device 144.

In some examples, an external sensor device may itself generate data in a format that may be applied to the ECG application electrode(s) and provided to the ECG application. However, in some examples, the external sensor device 128 may include an encoder, such as data encoder 154. The data encoder 154 may encode sensor data generated by the external sensor device 128 in a manner that may be applied to the electrode(s) 150 and received by an ECG application. For example, the data encoder 154 may include one or more frequency modulators as described herein.

The external sensor device 128 may include memory which may store sensor data for a period of time. In some applications, sensor data may be stored until transmission of the data is initiated by a user contact with one or more electrodes of the external sensor device 128 and/or the ECG electrodes electrode(s) 150.

Accordingly, during operation, external sensor device 128 may generate sensor data. The sensor data may in some examples be encoded, such as by data encoder 154. The sensor data may be communicated to the electronic device 144 by providing the sensor data to ECG circuitry 152, such as by providing signals encoding the sensor data to electrode(s) 150. In this manner, the electronic device 144 may generate an output from an ECG application, such as using executable instructions for ECG application 116. The output of the ECG application may be decoded to recover all or portions of the sensor data, for example in accordance with the executable instructions for sensor data decoding 148. In some examples, output of the ECG application may be transmitted to another device or system (e.g., another computer system) for decoding. The decoded sensor data may be displayed, analyzed, and/or otherwise acted upon.

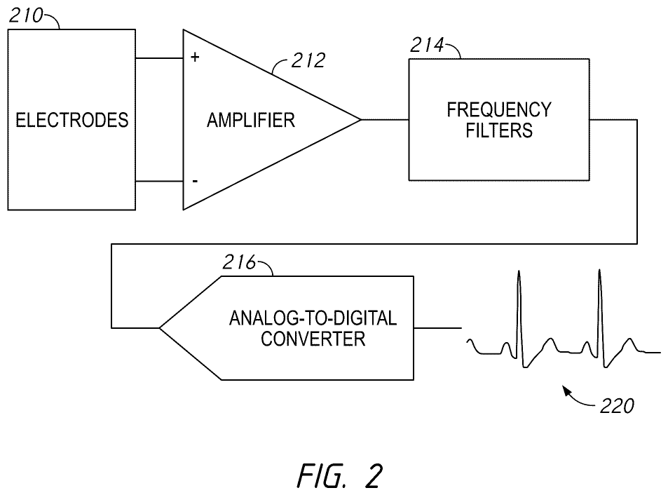

FIG. 2 is a schematic illustration of electrodes and ECG circuitry, arranged in accordance with examples described herein. The example of FIG. 2 includes electrodes 210, amplifier 212, filter(s) 214, and analog-to-digital converter 216. The ECG circuitry may include electrodes 210, amplifier 212, and filter(s) 214. The ECG circuitry may accordingly generate ECG signal 220. The example of FIG. 2 may be used to implement and/or may be implemented by components of FIG. 1. For example, the electrodes 210 may be used to implement and or may be implemented by electrode(s) 150 of FIG. 1. The ECG circuitry 152 of FIG. 1 may be used to implement and/or may be implemented by the amplifier 212, filter(s) 214, and analog-to-digital converter 216 of FIG. 2

The components shown in FIG. 2 are exemplary. Additional, fewer, and/or different components may be used in other examples.

Wearable devices with ECG functions (such as smartwatches) generally provide a single-lead ECG. This method, less comprehensive than a medical 12-lead ECG used in some clinical settings, still may offer valuable insights into the user's heart rhythm and rate. The ECG circuitry and electrodes of FIG. 2 provide an example of a single-lead ECG.

The electrodes 210 may be provided in various configurations in electronic devices described herein and used for ECG sensing. In some examples, one electrode may be integrated into the device and positioned for regular contact with a user of the device. For example, one electrode of the electrodes 210 may be integrated into a watch or other wearable device's back plate or other housing. The electrode may be in contact with the user, such as with the user's wrist. Another electrode of electrodes 210 may be positioned at another location of the electronic device, such as embedded on the side or on a button on a watch. In this manner, one electrode of the electrodes 210 is in constant contact with the user during normal operation. Another electrode may not be positioned for constant contact with the user when in use, but may be touched or otherwise brought into contact with the user. When an ECG recording is desired, the user may contact the second electrode (e.g., with an opposite hand). In this manner, a closed loop is formed across the heart, allowing the measurement of the heart's electrical signals. This setup is an example of a single-lead ECG. The electronic device measures the electrical potential difference between a constant contact point (e.g., the wrist) and another contact point (e.g., the finger) during a heartbeat, recording the heart's electrical activity.

The electrical activity may be represented as ECG signal 220. The ECG signal 220 may be represented by varying voltages and/or other parameters. The ECG signal 220 may be stored in memory described herein and/or may be displayed by devices described herein such as by display(s) 108 of FIG. 1 in accordance with executable instructions for ECG application 116.

Note that, generally, the amplifier 212 may be referred to as a high gain amplifier. The amplifier 212 may be suitable for amplifying potential differences caused by heartbeats, which may generally be referred to as small differences. Accordingly, the amplifier 212 may have a high gain. In examples described herein, the amplifier 212 may be used to amplify sensor data encoded in a signal, which sensor data may be encoded as small variations in a signal parameter.

Accordingly, in some examples, electrodes 210 and/or electrode(s) 150 of FIG. 1 may include one electrode positioned for continuous contact with a user during use of the device. Another electrode of electrodes 210 and/or electrode(s) 150 of FIG. 1 may be positioned for intermittent contact with a user during user of the device (e.g., positioned for a user to touch or otherwise contact a portion of their body with the other electrode).

In other examples, however, two electrodes of electrodes 210 and/or electrode(s) 150 may be positioned for continuous contact with a user during use of the device.

In other examples, two electrodes of electrodes 210 and/or electrode(s) 150 may be positioned for intermittent contact with a user during use of the device.

In some examples, ECG circuitry provided on electronic devices described herein, such as ECG circuitry 152 of FIG. 1 and/or the ECG circuitry shown and described with reference to FIG. 2, may generally be expected to capture input AC signals having a frequency between 0.1 Hz and 20 Hz in some examples, between 0.5 Hz and 10 Hz in some examples, and around 1 Hz in some examples (e.g., a frequency range around an expected frequency of a heart rate). ECG circuitry provided on electronic devices described herein may generally be expected to detect signal amplitudes of between 100 μV to 300 mV in some examples, between 500 μV to 100 mV in some examples. Generally, the ECG circuitry can be expected to detect amplitude variations comparable to those expected in an electrical signal generated due to a heartbeat.

Generally, ECG circuitry described herein may be able to differentiate frequencies separated by at least 0.05 Hz and may capture signals having frequencies of 10 Hz or less. Other frequencies and frequency separations may be used in other examples.

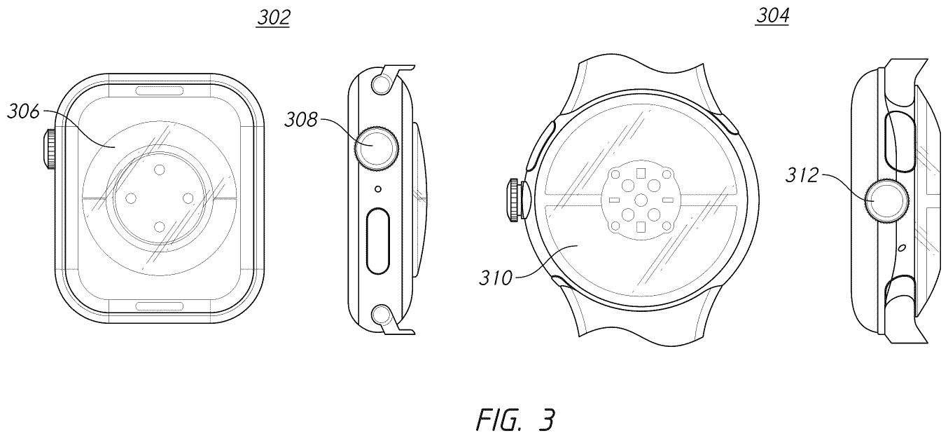

FIG. 3 is a schematic illustration of watches arranged in accordance with examples described herein. FIG. 3 illustrates a rear face and side view of watch 302 and a rear face and side view of watch 304. The watch 302 includes electrode 306 on a rear face and electrode 308 on a side of watch 302. The electrode 312 includes electrode 310 on a rear face and electrode 312 on a side of watch 304.

The watch 302 and watch 304 may be used to implement and/or implemented by the electronic device 144 of FIG. 1 in some examples. The electrode(s) 150 of FIG. 1 and/or electrodes 210 of FIG. 2 may be implemented, for example by electrode 306 and electrode 308 and/or electrode 310 and electrode 312 of FIG. 3.

During operation, the electrode 306 may be positioned to contact a user's wrist, for example. To take an ECG reading, a user may contact electrode 308 with a finger or other portion of their body. During operation of watch 304, the electrode 310 may be positioned to contact a user's wrist. To take an ECG reading, a user may contact electrode 312 with a finger or other portion of their body. The electrode 308 and electrode 312 are depicted on a crown of the respective watches. Other positions of the electrode 308 and/or electrode 312 are possible in other examples, such as on a face or side of the watches in some examples. The electrode 306 and electrode 310 are positioned on a back face of the watch, however, other positions and/or shapes are possible. In some examples, an electrode may be positioned on a watch band and may contact a user during regular use, for example.

Examples of systems described herein may provide a physical connection between one or more sensor devices and electrodes coupled to ECG circuitry described herein. Examples may generally provide a stable physical connection with an electronic device's ECG electrodes, one of which may be located on the back of the watch and the other on its crown, for example. Accordingly, sensor devices described herein, such as external sensor device 128 of FIG. 1 may include an electrode that is designed for continuous or intermittent connection with one or more of the ECG electrodes, such as electrode(s) 150.

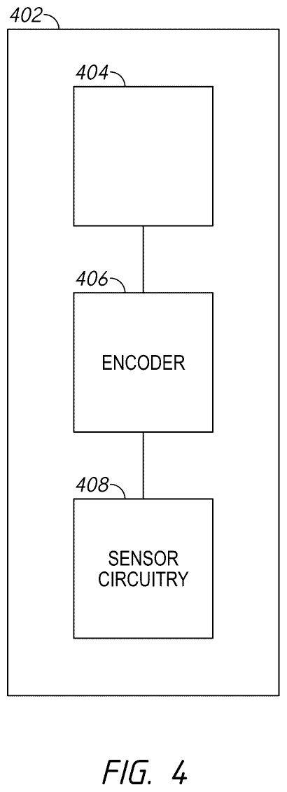

FIG. 4 is a schematic illustration of an external sensor device arranged in accordance with examples described herein. The external sensor device shown in FIG. 4 includes a substrate 402, an electrode 404, an encoder 406, and sensor circuitry 408. The external sensor device of FIG. 4 may be used to implement and/or implemented by sensor devices described herein, such as external sensor device 128 of FIG. 1.

The components of FIG. 4 are exemplary. Additional, fewer, and/or different components may be used in other examples.

Generally, an external sensor device may include an electrode, such as electrode 404 of FIG. 4. The electrode may be an area of a conductive material. The external sensor device may be shaped such that the electrode 404 is positioned for continuous or intermittent contact with one of the ECG electrodes described herein, such as the electrode(s) 150 of FIG. 1 and/or the electrodes 210 of FIG. 2.

The electrode 404 may be positioned on a substrate, such as the substrate 402. In some examples, the substrate 402 may be a flexible substrate. The flexible substrate may allow the external sensor device to be folded and/or shaped to bring the electrode 404 into contact with one or more ECG electrodes described herein. The flexible substrate may allow the external sensor device to be positioned such that a user may contact both the electrode 404 and one of the ECG electrodes with portions of the user's body. In this manner, a connection may be formed between the electrode 404 and one of the ECG electrodes using the user's body. In some examples, the substrate 402 may be a rigid substrate. For example, the substrate 402 may be formed as a case or connector or other structure suitable for mating or all or a portion of electronic device 144, such as to a face of a smartwatch. The electrode 404 may be positioned on the substrate in a manner to continuously and/or intermittently contact one of the ECG electrodes when the substrate is mated to all or a portion of the electronic device.

The sensor device may include sensor circuitry, such as sensor circuitry 408. Generally any circuitry may be included that may be used to sense or more parameters including one or more environmental parameters and/or one or more biological parameters. Examples of sensing circuitry include, but are not limited to, one or more temperature sensors, humidity sensors, accelerometers, gyroscopes, UV sensors, breath alcohol sensors, and/or touch button sensors. Other sensors may be used in other examples. Examples of sensors described herein may provide an output signal.

In some examples, the sensor circuitry 408 may include and/or may be coupled to a memory. The memory may store sensor data in some examples. The stored sensor data may be provided to ECG circuitry described herein at a later time. Accordingly, data may be collected and stored, and later communicated to an electronic device using an ECG interface.

Examples of external sensor devices described herein may include an encoder, such as the encoder 406 of FIG. 4. The encoder 406 of FIG. 4 may be implemented by and/or used to implement the data encoder 154 of FIG. 1, for example. The encoder 406 may be electrically coupled with the sensor circuitry 408. Accordingly, the encoder 406 may receive the sensor signals generated by one or more sensors. The encoder 406 may encode the sensor output in a manner suitable for providing to ECG circuitry described herein. For example, the encoder 406 may encode data received from sensor circuitry 408 using frequency modulation. Accordingly, the encoder 406 may output frequency modulated sensor data. The output from the encoder 406 may be suitable for providing to ECG circuitry. For example, the output may have amplitudes between 100 μV to 5 mV in some examples, between 500 μV to ImV in some examples. Generally, the output may have frequencies between 0.1 Hz and 20 Hz in some examples, between 0.5 Hz and 10 Hz in some examples, and around 1 Hz in some examples.

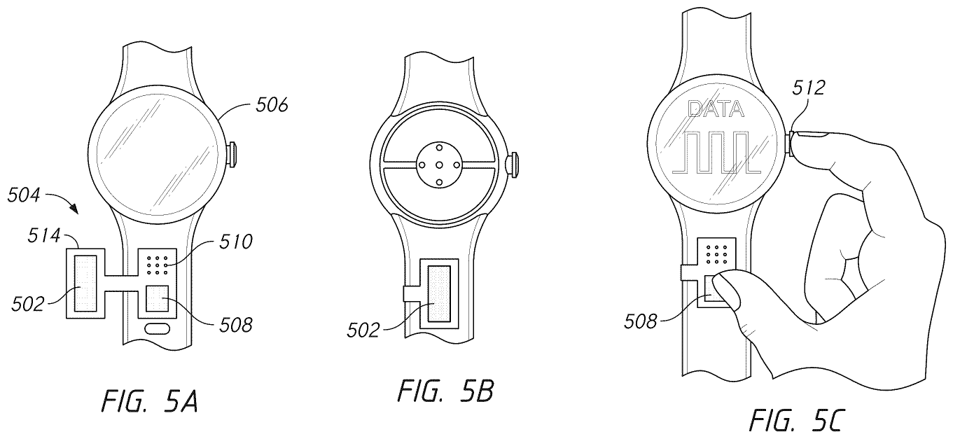

FIG. 5A, FIG. 5B, and FIG. 5C are schematic illustrations of a system arranged in accordance with examples described herein. In the example of FIG. 5A, FIG. 5B, and FIG. 5C, an external sensor device 504 is shown. The external sensor device 504 is coupled to electronic device 506, which is a smartwatch in the example of FIG. 5A, FIG. 5B, and FIG. 5C. The external sensor device 504 may be used to implement and/or may be implemented by sensor devices described herein, such as the external sensor device 128 of FIG. 1 and/or the external sensor device shown and described with reference to FIG. 4. The electronic device 506 may be used to implement and/or may be implemented by electronic devices described herein, such as the electronic device 144 of FIG. 1.

The external sensor device 504 includes substrate 514, electrode 502, electrode 508, and circuitry 510. The substrate 514 is flexible and arranged to support two electrodes-electrode 502 and electrode 508—on opposite sides of a portion of a watch band. The circuitry 510 may include sensor circuitry and an encoder as described herein. The circuitry 510 may include sensor circuitry and an encoder as described herein, such as the sensor circuitry 408 and encoder 406 of FIG. 4.

The substrate 514 may have an adhesive side opposite the circuitry. Accordingly, to couple the external sensor device 504 to the electronic device 506 in FIG. 5A, a portion of the substrate 514 may be adhered to a watch strap. The portion adhered to the watch strap may accordingly position electrode 508 to face a user during operation.

The substrate 514 may fold to adhere a second portion to an opposite side of the watch strap. In FIG. 5B, the substrate 514 is folded and adhered to the opposite side of the strap. The portion adhered to the opposite side of the strap supports electrode 502. Accordingly, electrode 502 may be positioned for continuous contact with a user of the electronic device 506 in some examples.

During operation, the circuitry 510 may generate sensor data. To communicate sensor data to the electronic device 506. In some examples, the sensor data may be stored in or by the circuitry 510. To communicate sensor data to the electronic device 506, a user may contact the electrode 508 and the ECG electrode 512 as shown in FIG. 5C. The sensor signals may travel through the user's body to reach the ECG circuitry. In the example of FIG. 5C, the ECG electrode 512 is located on a crown of the smartwatch used to implement the electronic device 506. The ECG electrode 512 may be located in other positions in other examples.

Accordingly, the circuitry 510 may generate sensor data (e.g., temperature sensor data, UV light data, touch button data, etc.). The circuitry 510 may further encode the sensor data using, for example, frequency modulation. When the electrode 508 is coupled to an ECG electrode of the electronic device 506 (e.g., by a user contacting both the electrode 508 and the ECG electrode 512), the encoded sensor data may be provided to ECG circuitry of the electronic device 506.

Note also that the external sensor device of FIG. 5A-FIG. 5C may be utilized with multiple electronic devices. For example, it may be suitable for use with a number of smartwatches. A user may attach the external sensor device to one electronic device, use it, and then remove the external sensor device and attach it to a different electronic device (e.g., a different smartwatch). In this manner, the external sensor devices described herein may be re-usable and/or interchangeable.

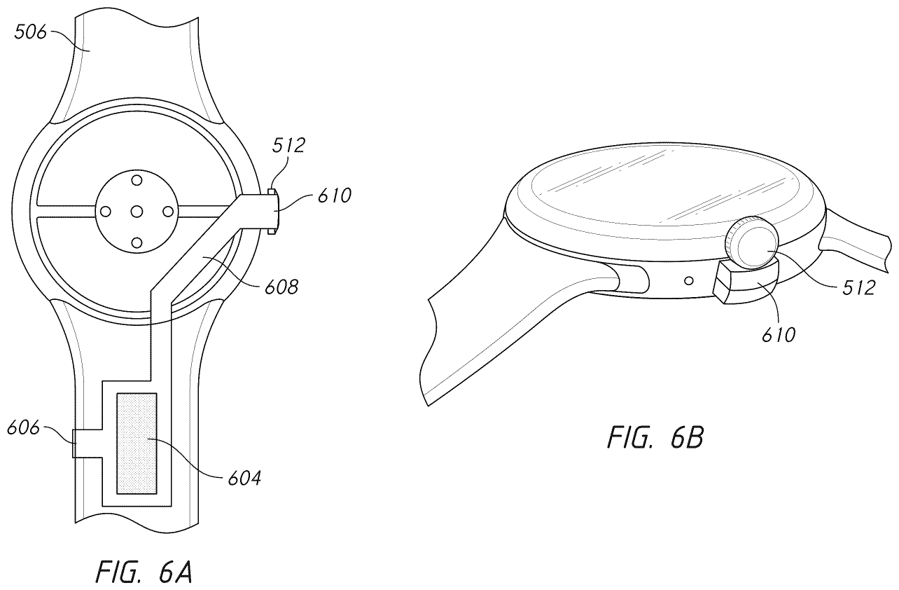

FIG. 6A and FIG. 6B are schematic illustrations of a system arranged in accordance with examples described herein. The system depicted in FIG. 6A and FIG. 6B includes another example of an external sensor device coupled to an electronic device. The electronic device shown in FIG. 6A and FIG. 6B, electronic device 506, is the same as that depicted in FIG. 5A-FIG. 5B. The external sensor device shown in FIG. 6A and FIG. 6B may have analogous components to those shown in FIG. 5A-FIG. 5C, however the external sensor device shown in FIG. 6A and FIG. 6B may be configured for continuous contact with ECG circuitry of the electronic device 506.

The system of FIG. 6A-FIG. 6B may be used to implement and/or may be implemented by systems described herein, such as the system shown and described with reference to FIG. 1. For example, the electronic device 506 may be used to implement and/or implemented by the electronic device 144 of FIG. 1. The external sensor device of FIG. 6A-FIG. 6B (including the substrate 606, electrode 604, and electrode 610), may be used to implement and/or be implemented by the external sensor device 128 of FIG. 1 and/or the external sensor device shown and described with reference to FIG. 4.

In the example of FIG. 6A, the external sensor device may include circuitry (e.g., sensor circuitry and/or an encoder) on one side of a substrate 606, such as the circuitry 510 of FIG. 5A. The sensor circuitry and/or encoder may be provided in other positions and coupled to the described electrodes in other examples. One electrode, electrode 604 is positioned on an opposite side of the substrate 606 and may be positioned on a portion of the electronic device 506 (e.g., on a strap of a smartwatch) for continuous contact with the user. In the example of FIG. 6A, the substrate 606 includes an extension portion 608. The extension portion 608 is provided to position another electrode, electrode 610, in contact with an ECG electrode of the electronic device 506. Accordingly, the extension portion 608 is provided across a back face of the smartwatch of FIG. 6A such that electrode 610 is positioned in contact with ECG electrode 512. The extension portion 608 is angled across the back face to avoid disrupting any additional existing sensors (e.g., photoplethysmogram (PPG), SpO2, and/or electrodermal activity (EDA)). In the example of FIG. 6A, the electronic device 506 includes ECG electrode 512 in a crown of the smartwatch.

FIG. 6B is a side view of the electronic device 506 and external sensor device of FIG. 6A. In the side view, the electrode 610 may be seen in contact with ECG electrode 512 which is in the crown of the smartwatch used to implement the electronic device 506. Accordingly, the extension portion 608 may include a fixture supporting an electrode. The fixture may be positioned to make contact with the watch crown. In this manner, the electrode 610 may make sliding contact with the ECG electrode 512 (e.g., the watch crown). An example fixture may include a 3D printed material. The 3D-printed part may include a conductive surface (e.g., electrode 610) supported by soft foam to make reliable ohmic contact with the crown. This example may make sufficient contact with the crown without hindering the crown's functionality for the user in its use as a watch crown.

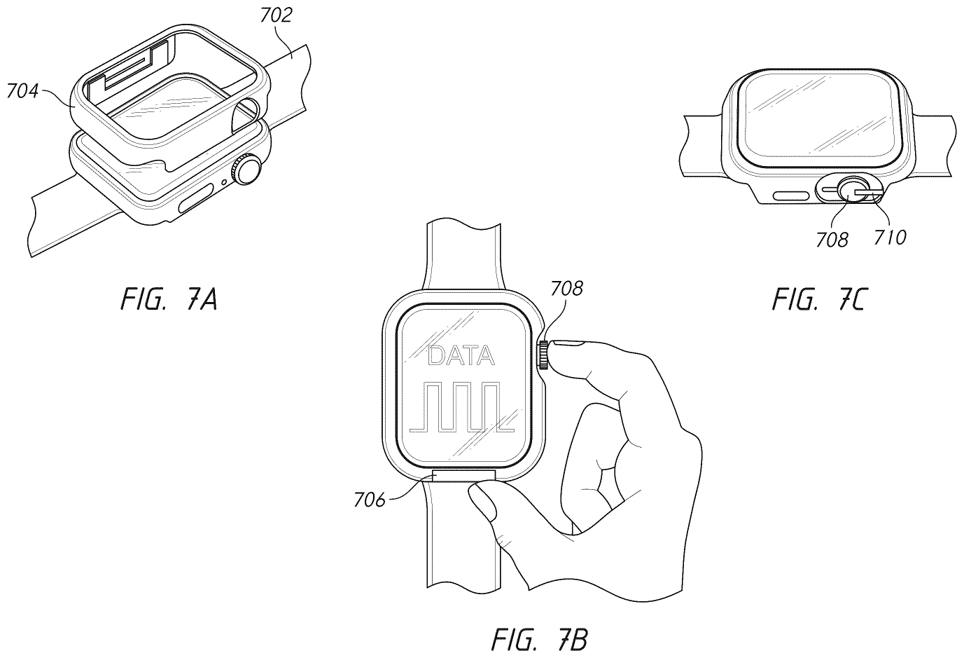

Examples of external sensor devices may be provided as a case for an electronic device, or for a portion of an electronic device. FIG. 7A-FIG. 7C are schematic illustrations of a system where an external sensor device is integrated into a case for a face of a smartwatch. However, other cases are possible—e.g., a case for a smartphone, a case for earbuds or other headphones, a case for a smartspeaker, a case or other attachment for a ring, etc.

The system of FIG. 7A-FIG. 7C may be used to implement and/or may be implemented by systems described herein, such as the system shown and described with reference to FIG. 1. For example, the electronic device 702 may be used to implement and/or implemented by the electronic device 144 of FIG. 1. The external sensor device of FIG. 7A-FIG. 7C may be used to implement and/or be implemented by the external sensor device 128 of FIG. 1 and/or the external sensor device shown and described with reference to FIG. 4.

In FIG. 7A, a case 704 is depicted for electronic device 702, a smartwatch. The case 704 may snap onto or otherwise attach to a face of the smartwatch. The case 704 may include integrated components of an external device described herein. For example, electrode 404, sensor circuitry 408, and/or encoder 406 of FIG. 4 may be integrated into case 704. In this manner, when the case 704 is attached to electronic device 702, the electronic device 702 is supporting, carrying, or otherwise attached to the external sensor device. Note that the case 704 may be removed from the electronic device 702 in some examples. The case 704 may be placed on other electronic devices (e.g., other smartwatches) in some examples. Accordingly, the external sensor device implemented in case 704 may be detachable and reusable.

Accordingly, users may attach or detach sensors from the electronic device 702 simply by changing the case. This approach provides flexibility to switch watch bands without needing to replace the sensors. To install the case 704, users may snap it onto the watch. In some examples, the case 704 includes or is coupled to a ground contact (e.g., a conductive strip). In some examples, a user may attach the ground contact to the watch's back or other ground provided by the electronic device 702.

Examples of external sensor devices incorporated into cases may also accommodate both continuous and manual activation sensing.

FIG. 7B is a schematic illustration of an external sensor device integrated into a case and used in manual activation sensing. For manual activation, an electrode 706 may be integrated into case 704. The electrode 706 may be accessible to the user and may be coupled to other components of the external sensor device, such as sensor circuitry and/or an encoder. The electrode 706 of FIG. 7B is positioned on an edge of the case, facing the user. To transmit sensor data to the electronic device, a user may contact both the electrode 706 and an ECG electrode of the electronic device 702 (e.g., an ECG electrode in the crown, such as ECG electrode 708). Accordingly, a user may use one hand to contact both the electrode 706 on the case and the ECG electrode 708 on the crown of the electronic device 702.

Sensor data generated by the external sensor device, such as by sensor circuitry integrated in the case 704 may be encoded (e.g., by one or more encoders). The encoded sensor data may be provided to the ECG electrode 708 through the user's body when contact with electrode 706 and ECG electrode 708 are made. The encoded sensor data may be received and/or decoded by an ECG application on the electronic device 702 (e.g., in accordance with executable instructions for ECG application 116). The sensor data may be displayed on a display of the electronic device 702 (e.g., on the display(s) 108 of FIG. 1).

FIG. 7C is a schematic illustration of an external sensor device integrated into a case and used in continuous sensing. For continuous sensing, the case 704 may maintain constant electrical contact with an ECG electrode of the electronic device 702 (e.g. ECG electrode 708). The case 704 includes a conductive projection 710 (e.g., a gold finger) on the side of the case 704. This projection may make sliding contact with the ECG electrode (e.g., with the ECG electrode 708). Examples may make an ohmic connection without hindering the crown's functionality for the user in its use as a watch crown.

Some examples of external sensor devices may not incorporate a mechanical connection to the electronic device. Such examples may be referred to as companion sensors.

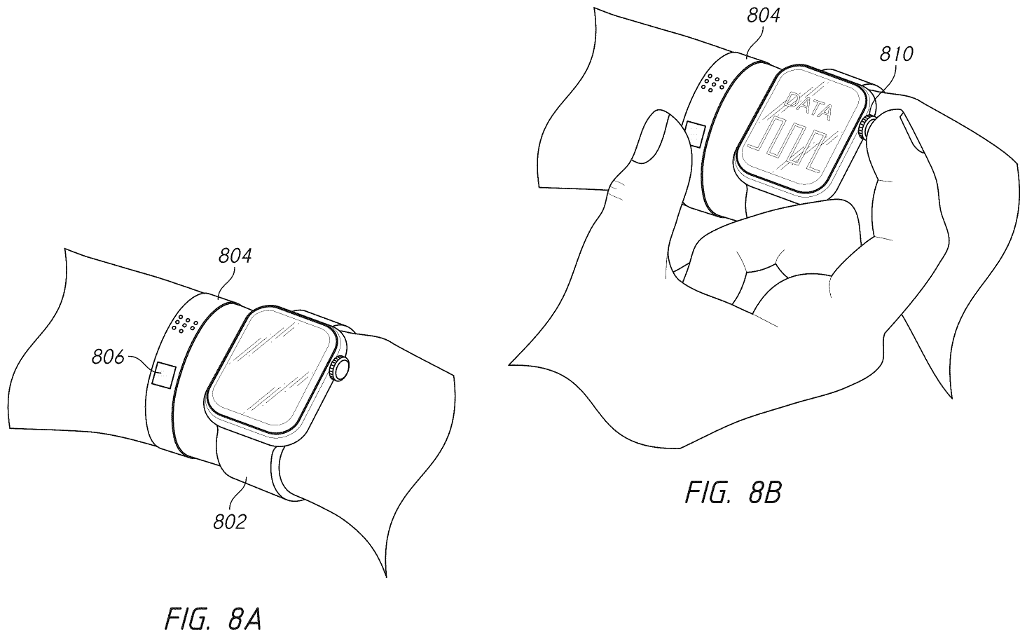

FIG. 8A is a schematic illustration of a system arranged in accordance with examples described herein. FIG. 8A depicts electronic device 802 and external sensor device 804. The electronic device 802 is depicted as a smartwatch, although other electronic devices may be used. The electronic device 802 may be implemented by and/or used to implement the electronic device 144 of FIG. 1. The external sensor device 804 may be implemented by and/or used to implement external sensor devices described herein, such as external sensor device 128 of FIG. 1 and/or the external sensor device shown and described with reference to FIG. 4.

Accordingly, the external sensor device 804 may include sensor circuitry, one or more encoders, and electrode 806. In the example of FIG. 8A, the external sensor device 804 is implemented using a flexible substrate that is worn around a user's wrist. Other implementations may be used in other examples, such as a substrate worn around an ankle, head, chest, or finger. In some examples, the substrate used to implement the external sensor device 804 may not be worn or carried by the user, but may be substrate in an environment of the user (e.g., on a wall, table, desk, floor, and/or ceiling). The electrode 806 electrically coupled to sensor circuitry may be accessible to the user.

To activate sensing, as shown in FIG. 8B, a user may contact both the electrode 806 provided by the external sensor device and an ECG electrode 810 of the electronic device 802. In this manner, sensor data encoded by the external sensor device may be provided to the ECG interface of the external sensor device 804. The sensor data may be displayed on a display of the external sensor device 804. Accordingly, data transfer is achieved by the user touching an exposed electrode on the external sensor device with one finger and the crown of the smartwatch with the other.

Accordingly, companion sensors may provide add-on sensors without the need to attach them directly mechanically to a watch or other electronic device. Instead, the sensor can be positioned on a separate band or accessory and can be placed anywhere, not just on the wrist. A companion sensor implementation may be particularly advantageous for sensors that are used on an occasional or periodic basis, such as a breath alcohol sensor that can be worn before going to a social gathering or a smart bandage with sensors designed to track the progress of wound healing.

Examples of external sensor devices described herein may incorporate any of a variety of sensor circuitry to sense a variety of environmental and/or physical parameters. Examples of UV light sensors, body temperature sensors, breath alcohol sensors, and touch buttons are described. However, it is to be understood that other sensor circuitry may be used in other examples. The sensor circuitry described may, for example, be included in external sensor device 128 of FIG. 1. The sensor circuitry described may be used to implement sensor circuitry 408 of FIG. 4. The sensor circuitry described may be used to implement circuitry 510 of FIG. 5A. The sensor circuitry described may be incorporated in case 704 of FIG. 7A. The sensor circuitry described may be included in external sensor device 804 of FIG. 8A.

In some examples, a UV light sensor may be used as the sensor circuitry (e.g., sensor circuitry 408 of FIG. 4, circuitry 510 of FIG. 5A). UV light exposure can cause severe skin damage, such as sunburn, early signs of aging, and an increased risk of skin cancer. Prolonged exposure to UV rays can also harm the eyes, potentially leading to conditions like cataracts, and may suppress the immune system, reducing the body's ability to fend off certain infections. Therefore, monitoring UV exposure, especially when outdoors, may be advantageous to minimize these health risks.

UV radiation is measured using the UV Index, which quantifies the risk of sunburn from UV rays at a specific location and time. It is designed to help people understand the potential for skin and eye damage from the sun's UV rays and to indicate precautions to take. Higher numbers indicate a greater risk of harm and a need for sun protection. Sensor circuitry described herein may include circuitry which may output the UV index.

For example, the case 704 of FIG. 7A may include integrated sensor circuitry to continuously measure and communicate UV index values. The sensor may be built as a flexible printed circuit board (PCB) with a layout that fits entirely in the case 704. The case 704 may make contact with an ECG electrode via a conductive projection 710 and has a flexible electrode to make contact with the watch's ground (e.g., a backside of the watch case). A nano-level output of the UV sensor circuitry may be is amplified by an op-amp amplifier and frequency modulated by a timer using an encoder (e.g., encoder 406) and provided to the ECG circuitry of the watch. The external sensor device may in some examples be powered by ambient light, from which energy is harvested using photodiodes.

In this manner, sensor data encoding UV index values may be provided to ECG circuitry of electronic devices described herein. ECG applications may be utilized to access the sensor data, which may be displayed on the electronic device, transmitted to another electronic device, and/or decoded using decoder circuitry and/or software (e.g., executable instructions for sensor data decoding 148) described herein.

In some examples, a body temperature sensor may be used as the sensor circuitry (e.g., sensor circuitry 408 of FIG. 4, circuitry 510 of FIG. 5A). Some electronic devices, such as the Apple Watch Series 8 and Garmin models, integrate skin temperature sensors into their health monitoring features. Though they can provide valuable health insights, body temperature sensor readings are less accurate for measuring core body temperature than methods like forehead or ear thermometers because readings at extremities like the wrist can vary due to external factors. This concern may be particularly relevant for detecting fever or other medical conditions, where precise temperature readings may be advantageous.

Accordingly, it may be advantageous to provide external sensor devices that can provide more precise temperature measurements. These add-ons can complement electronic devices such as smartwatches, offering a more reliable solution for temperature monitoring for health purposes.

An example of body temperature sensor circuitry may be used, for example as a watch strap sensor. For example, the circuitry 510 of FIG. 5A may include body temperature sensor circuitry. The external sensor device may attach to one side of a watch strap and wraps around it to establish skin contact for ground connection. As shown in FIG. 5A. The circuitry 510 may be tailored for on-demand temperature measurement and equipped with electrode 508 that lets the user make contact to initiate communication with the watch crown, such as shown in FIG. 5C. Once the sensor is added to the watch, users can hold it against their forehead, such that the forehead makes contact with a portion of the circuitry 510, to take a temperature reading. To transfer this data to the watch, the user simply needs to establish a connection between the watch and sensor with their fingers, such as shown in FIG. 5C.

An example temperature sensor circuitry may generate an analog voltage indicative of body temperature. The analog voltage may be amplified by an amplifier included in the sensor circuitry and/or the external sensor device. An encoder may be used to frequency modulate the amplified analog voltages. Upon user connection, the frequency modulated data may be transmitted to an electronic device, such as electronic device 506 of FIG. 5C. The sensor's circuitry may be powered by a coin cell battery. To conserve battery life, the battery may be normally disconnected and may become active through a switch. Users can activate the circuit by pressing down on the switch to activate the circuit.

In some examples, pressing the button to activate the circuit may not be used during measurement since the measurement process may not require battery power. Instead, the sensor circuitry may operate by the body warming the temperature sensor to reach thermal equilibrium. The battery power may be connected to transmit data to the watch.

In some examples, an alcohol breath sensor may be used as the sensor circuitry (e.g., sensor circuitry 408 of FIG. 4, circuitry 510 of FIG. 5A). An alcohol breath sensor, commonly known as a breathalyzer, generally refers to a device for measuring Blood Alcohol Content (BAC) from a person's breath. BAC is typically measured as a percentage of alcohol in the bloodstream. Breathalyzer devices work by assessing a chemical reaction with alcohol, leading to a color transition or an alteration in electrical signals. Example breathalyzer sensors may be based on semiconductor oxide and fuel cell technology. To use this sensor, an individual blows into or across the sensor circuitry, which then measures the alcohol level in the exhaled breath, converting it into a BAC reading.

Example alcohol breath sensor circuitry may be used and included in external sensor devices described herein. A user may wear the external sensor device on a wearable device (e.g., a smartwatch) or may remove the external sensor device and utilize it independently. An example external sensor device includes an electrode (e.g., electrode 610 of FIG. 6A) to establish a connection with an ECG electrode (e.g., ECG electrode 512). The ECG electrode may be located on a smartwatch's crown. The external sensor device may include another electrode on the opposite side to maintain a ground connection against the skin (e.g., electrode 604).

An example external sensor device includes a Metal Oxide (MOX) gas sensor, which detects alcohol levels and outputs them as an analog voltage. This output may be amplified by an op-amp amplifier and then frequency modulated using an encoder, such as encoder 406. The circuit may be powered by a compact 3 mAh battery, which may be activated via a switch. The switch may be positioned, for example, under an electrode used to make finger contact with a user (e.g., electrode 508 of FIG. 5C). This switch can be conveniently pressed at the same time as the finger establishes a connection with the ECG electrode in the watch's crown. The gas sensor may be a main driver of the circuit's power consumption. This allows the battery to power multiple breath measurements, each of which may last 20 seconds in some examples.

In some examples, a touch button may be used as the sensor circuitry (e.g., sensor circuitry 408 of FIG. 4, circuitry 510 of FIG. 5A). Smartwatches or other electronic devices, with their compact screens, often present challenges for user input. Limited screen space not only makes precise touch interactions difficult, especially for those with larger fingers, but also restricts the addition of extra control elements like buttons. Though voice control offers a solution, it can raise privacy concerns and is not always the most convenient in public settings.

Examples of external sensor devices described herein may include one or more additional buttons. The buttons may be positioned, for example on a watch strap, such as in circuitry 510 of FIG. 5A. For example, four buttons may be provided in one example to form a directional pad (D-pad). Users can navigate via this D-pad, with each button doubling as a shortcut for quick access to functions. Furthermore, they can use these buttons in combination with other watch features, like the crown, to enhance existing interactions. For instance, a button could modify the crown's scrolling speed or access different menus, optimizing the user experience without compromising the watch's compact design.

The buttons may be powered by ambient light using photodiodes in some examples. Additionally, a storage capacitor may be provided to store charge, ensuring functionality even in the absence of ambient light.

In some examples, the output of the touch button(s) may be encoded using frequency control achieved by altering feedback from an output of an op-amp to its negative terminal. Each button press enables an alternate feedback path with separate resistance, resulting in different oscillator output frequency. This frequency resolution may be adequate to distinguish four discrete frequencies corresponding to four button presses in the example of a D-pad.

The circuit may operate continuously using energy harvested from photodiodes.

While examples of single functionality sensors have been described herein, it is to be understood that in some examples multiple sensor functionalities may be used, and may be operating simultaneously. For example, the external sensor device 128 of FIG. 1, and/or the external sensor device shown and described with respect to FIG. 4 may include sensor circuitry able to sense multiple parameters (e.g., blood alcohol level and UV light). Any number of parameters may be sensed. The sensing circuitry for multiple parameters may be integrated and/or may be provided as separate circuitry in external sensor devices described herein. In some examples, encoders described herein may utilize distinct carrier frequencies for each sensor circuitry's data stream. In some examples, a frequency resolution of 0.05 Hz within a total bandwidth of 0.5 to 10+Hz provides for multiple concurrent sensor channels.

External sensor devices described herein such as UV light detectors and buttons on a smartwatch can interfere with ECG readings by transmitting data that overlap with the frequency band of interest for ECG readings. To mitigate this, in some examples, sensor operations may be temporarily halted during ECG capture. In some examples, a switch may be provided on the external sensor device which may pause (e.g., turn off) the transmission from the external sensor device. In this manner, a user could activate the switch when the ECG electrodes were to be used to take ECG reading(s). In some examples, external sensor devices described herein may utilize encoders which may encode the sensor data into frequencies generally outside the ECG signals' frequency range (0.5 Hz to 150 Hz). For example, sensor data may be encoded using frequency modulation in frequencies less than 0.5 Hz and/or greater than 150 Hz in some examples.

In some examples, energy harvesting has been described, such as energy harvesting from ambient light. In other examples, other energy-harvesting methods could be employed, including generating power through watch vibrations by activating the vibration motor, capturing sound energy via speaker output, or collecting energy during the electronic device's charging process for storage in a supercapacitor or battery. These examples may allow the external sensor device to operate independently of ambient conditions, ensuring continuous availability in some examples.

Some examples described herein utilized bare flex printed circuits. To function in the same environments as wearable devices, more robust mechanical integration may be used in some examples. For example, injected molded parts may be used to gain water and dust resistance.

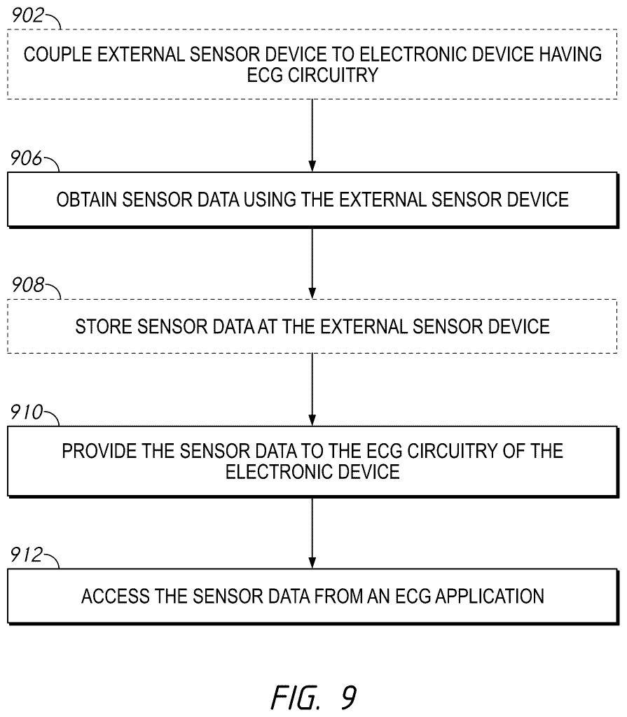

FIG. 9 is a flowchart illustrating an example method arranged in accordance with examples described herein. FIG. 9 includes block 902, “couple external sensor device to electronic device having ECG circuitry.” Block 902 may be followed by block 906, “obtain sensor data using the external sensor device.” Block 906 may be followed by optional block 908, “store sensor data at the external sensor device.” Block 908 may be followed by block 910, “provide the sensor data to the ECG circuitry of the electronic device.” Block 910 may be followed by block 912, “access the sensor data from an ECG application.”

It is to be understood that the blocks shown in FIG. 9 are exemplary. Additional, fewer, and/or different blocks may be used in other examples, and the blocks may be performed in other orders.

The method described with respect to FIG. 9 may be performed using systems described herein, such as the system shown and described with reference to FIG. 1.

Block 902 recites “couple external sensor device to electronic device having ECG circuitry.” For example, a user may mechanically couple an external sensor device to an electronic device. The electronic device may then wholly or partially support the external sensor device, and/or the external sensor device may move through an environment with the electronic device. An external sensor device may be adhered to, affixed to, clipped to, wrapped around, snapped onto, or otherwise coupled to an electronic device. For example, in FIG. 5A the external sensor device 504 may be adhered to the electronic device 506 by adhering substrate 514 to a band of a smartwatch. As another example, in FIG. 7A, an external sensor device may be integrated in a case 704, and the case 704 may be coupled to the electronic device 702 by snapping the case 704 to a face of the smartwatch.

Note that, in some examples, the external sensor device may not be mechanically coupled to the electronic device. For example, the external sensor device 804 of FIG. 8A may not have a mechanical connection to the electronic device 802. Accordingly, block 902 may not be performed in some examples.

Block 902 may be followed by block 906 and/or block 906 may be performed without block 902. Block 906 recites “obtain sensor data using the external sensor device.” Sensor data may be obtained using external sensor devices described herein, such as external sensor device 128 of FIG. 1, the external sensor device shown and described with respect to FIG. 4, and/or any of the external sensor devices shown in FIG. 5A-FIG. 8B. Generally, sensor circuitry described herein will be used to generate the sensor data. Any of a variety of sensors and/or sensor circuitry may be used including, but not limited to one or more UV sensors, temperature sensors, humidity sensors, pH sensors, accelerometers, gyroscopes, breath alcohol sensors, wound sensors, and/or touch buttons. In some examples, external sensor devices may include and/or be coupled to an input/output device (e.g., a button, touchscreen, or other circuitry). The input/output device may be used to initiate data capture in some examples. Accordingly, to start generating sensor data in some examples, a user may provide an indication through an input device. In some examples, no indication is provided and an external sensor device may generate sensor data without user input.

Block 906 may be followed by optional block 908. Block 908 recites “store sensor data at the external sensor device.” In some examples, sensor data may be stored, such as in a memory device included in and/or coupled to the sensing circuitry of the external sensor device. In some examples, sensor data may be communicated to another device (e.g., an electronic device) as it is generated, and storage may not be used. Accordingly, block 908 may be optional.

Block 908 (and/or block 906) may be followed by block 910. Block 910 recites “provide the sensor data to the ECG circuitry of the electronic device.” The sensor data, either as generated, and/or as stored, may be provided to ECG circuitry of an electronic device. Generally, this occurs by providing an electrical connection between an electrode of an external sensor device and an ECG electrode. For example, an electrode of the external sensor device 128 of FIG. 1 may contact one of the electrode(s) 150. The electrode 404 of FIG. 4 may be brought into electrical communication with one or more of the electrode(s) 150. Other examples of electrical communication between external sensor devices and ECG electrodes may occur, including as described herein. In some examples, the electrical communication may occur through all or a portion of a user. For example, a portion of a user's body may contact both an electrode of the external sensor device and an ECG electrode. For example, FIG. 7B depicts a user contacting electrode 706 and ECG electrode 708. In this manner, the user facilitates electrical connection between the external sensor device and the ECG electrode. In some examples, the sensor data may be encoded for transmission to the ECG circuitry. For example, frequency modulation may be used to provide the sensor data encoded using amplitudes and/or frequencies compatible with the ECG circuitry. While block 910 describes the transmission of sensor data, in some examples, a switch or other user input device may be used to pause, interrupt, and/or prevent data from being transmitted from the external sensor device, such as when an ECG reading is being made.

Block 910 may be followed by block 912. Block 912 recites “access the sensor data from an ECG application.” Electronic devices described herein may include ECG applications, such as executable instructions for ECG application 116 of FIG. 1. The ECG application may facilitate access to data presented to the ECG circuitry. For example, as described herein an ECG application may generate and/or access a digital data stream representing an analog input to one or more ECG electrodes. In some examples, the sensor data may be accessed by accessing the ECG application. The data stream generated by the ECG application may be provided to another application, such as a sensor data decoding application (e.g., in accordance with executable instructions for sensor data decoding 148 of FIG. 1). The sensor data decoding application may decode the sensor data from the data stream received from the ECG application (e.g., demodulate the data). The sensor data may be displayed and/or further analyzed. Decode circuitry may additionally or instead be used in some examples. The sensor data may be decoded at the electronic device and/or the sensor data may be communicated (e.g., through wired or wireless communication) to one or more other computer systems for decoding and/or analysis.

From the foregoing it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

What is claimed is:

1. A method comprising:
providing sensor data from an external sensor device to an ECG application; and
decoding an output of the ECG application to obtain the sensor data.

2. The method of claim 1, wherein said providing data from the external sensor device to the ECG application comprises providing the sensor data through an electrode configured for use by the ECG application.

3. The method of claim 1, wherein the ECG application is configured for operation on a wearable device.

4. The method of claim 1, wherein said providing data comprises encoding the sensor data from the external sensor device to provide the sensor data to the ECG application.

5. The method of claim 1, wherein said encoding comprises frequency modulation.

6. The method of claim 1, further comprising contacting at least one electrode on a wearable device configured for use by the ECG application with at least one electrode coupled to a sensor configured to generate the sensor data.

7. The method of claim 6 wherein the at least one electrode coupled to the sensor is implemented in a flexible printed circuit board, a case, or combinations thereof configured to couple to the wearable device.

8. The method of claim 1, further comprising contacting at least one wearable device electrode configured for use by the ECG application with a portion of a body of a user and contacting at least one electrode coupled to a sensor configured to generate the sensor data with a portion of the body of the user.

9. The method of claim 1, wherein the external sensor device comprises a UV light sensor, a body temperature sensor, a breath alcohol sensor, a touch button, or combinations thereof.

10. A system comprising:
an electronic device including:
ECG circuitry;
at least one ECG electrode coupled to the ECG circuitry;
an external sensor device comprising:
sensor circuitry configured to generate sensor data;
an encoder coupled to the sensor circuitry and configured to encode the sensor data for the ECG circuitry;
at least one electrode coupled to the encoder and configured to couple to the at least one ECG electrode.

11. The system of claim 10, wherein the external device further comprises a memory configured to store the sensor data.

12. The system of claim 10, wherein the external sensor device comprises a switch configured to pause transmission of the sensor data to the ECG electrode.

13. The system of claim 10, wherein the external sensor device includes a case configured to snap onto a portion of the electronic device, wherein the sensor circuitry, encoder, and the at least one electrode are integrated into the case.

14. The system of claim 10, wherein the external sensor device is positioned for a user to simultaneously contact the at least one electrode and the at least one ECG electrode to transmit the sensor data.

15. The system of claim 10, wherein the external sensor device comprises a flexible substrate configured to support the sensor circuitry, the encoder, and the at least one electrode.

16. The system of claim 15, wherein the flexible substrate includes an extension portion, wherein the electronic device comprises a smartwatch, and wherein the extension portion is configured to position the at least one electrode in contact with a crown of the smartwatch.

17. The system of claim 15, wherein the electronic device comprises a smartwatch and wherein the external sensor device is configured to attach to a band of the smartwatch.

18. The system of claim 15, wherein the external sensor device further comprises a ground electrode.

19. The system of claim 15, wherein the electronic device further comprises at least one non-transitory computer readable medium encoded with instructions which, when executed, cause the electronic device to obtain the sensor data from an ECG application.

20. The system of claim 15, wherein the electronic device comprises a smartwatch and the external sensor device comprises a companion sensor device, the companion sensor device configured to be worn around a wrist of a user of the smartwatch.
